SEQUENCE LISTING (22 sequences)

SEQ ID NO: 1 - DNA, 600 bp, Artificial Sequence
  NtPR1a terminator DNA sequence
ttgaaacgacctacgtccatttcacgttaatatgtatggattgttctgcttgatatcaagaacttaaataattgctctaaaaagcaacttaaagtcaagtatatagtaatagtactatatttgtaatcctctgaagtggatctataaaaagaccaagtggtcataattaaggggaaaaatatgagttgatgatcagcttgatgtatgatctgatattattatgaacacttttgtactcatacgaatcatgtgttgatggtctagctacttgcgatattacgagcaaaattcttaactacatgccttaggaacaagcttacacagttcatataatctactagagggccaaaaacatgaaaattaccaatttagatggtaggaggatattgaaagtggagcagctagttttaataactgaccgttagtcttaaaattgacggtataaaaatatttacataatcaggtcatttataaggtaattataggtaaatatttatgacgaattctcaatagtaatctgaaaaaaaattgtaactaacctattatactaaaactactataataggttagattacattaatcatgtcattagaagatctt

SEQ ID NO: 2 - DNA, 600 bp, Artificial Sequence; identical to SEQ ID NO: 1
  NbPINII terminator DNA sequence

SEQ ID NO: 3 - DNA, 634 bp, Artificial Sequence
  NbWIN1 terminator DNA sequence
gaagctatatacatatatatggccatgtttaccctttgacggcccaaataaaagtaaaagaacgatatgtaaaaggaaaaagaaaataaagttgctttgattgggttatgcaattccaatatctattcaagaattaatgtctttcgtttgggaagaatgaggtgaagtgtgtattatctttgtgattttaaataaaaaaatcacagtgggacagtatttgtgattatattaggttgctgttgttgtattttagtagttattccttttagtttttacccaacaccccaatttgcaaaggattgcacaagctgagttcacacctgctacttccaaacacaaaatgtcatctacataagacaactaatgtatataaagcaaaaatagaactaccatcttttggaagagatgtaactagaactagctaggaaataataacttggaatatcagaagaatgtattccctaggttcgatggatgtttgtattattgctagaacatattacatagattatgaatattaaaataagcgcaggcagattttgactgaaattctgaatggataggactctggaacattctcaatagtgagggagttcttttttttttttttttttttttaacttaactgtcaact

SEQ ID NO: 4 - DNA, 633 bp, Artificial Sequence
  NbPDF1.2 terminator DNA sequence
taattgaaattggattgatgctatgtatgcccaaaaaagggagatatgtgaagtttgttataataaggcgagagatgtaaattacaccaataggtgtaagacttctttatattctcggtatgtataacttaaatcatcgtgatagtgcactcaaactaagtctcttccataagaataacaaataaataaataaataaataaaatctttttttctttttgtccaagaaaataaataaaaactccacatacgcgacacgattgccttgacaaatggaagaatattgaaggtagcatgcatgtgaggcaatcttgacgacttcaaaaacttcttaagatgttgaatggcatcagaaataacattttagtatgtgtttacataagtttcatcactttgcctaaaaacaaggaagttgtgagttcgagtcattctaagagcaaggtggagagttcttcaaaggaagaatgtcggggatctatttggaaacaacctatctaccctatgatacgggtaaggtttgcgtatacactattcttccagatcccacttttaaaattatactagcttgttattatatttaattatgttataaatttaagtaattaaagaaagacttttgacatctaagtctagca

SEQ ID NO: 5 - DNA, 535 bp, Artificial Sequence
  StPINII terminator DNA sequence
ccctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgatta

SEQ ID NO: 6 - DNA, 551 bp, Artificial Sequence
  Tnos terminator DNA sequence
agaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcaaacttcggcactgtgtaatgacgatgagcaatcgagaggctgactaacaaaaggtatgcccaaaaacaacctctccaaactgtttcgaattggaagtttctgctcatgccgacaggcataacttagatattcgcgggctattcccactaattcgtcctgctggtttgcgccaagataaatcagtgcatctccttacaagttcctctgtcttgtgaaatgaactgctgactgccccccaagaaagcctcctcatctcccagttggcggcggctg

SEQ ID NO: 7 - DNA, 1922 bp, Artificial Sequence
  AtMAR1a DNA sequence
agagttgccggtgcaactcctcccataccaaaatagtgttttatcttatttttaaaatagataactttttcaacttatttgtttaaaattttagcttacgttacatgttataataataattaaaaaaagaagaagatatagtaataacatttgagaagttgcatttgggttccatgtttatacattcttcacttctaatgaataacaccttgaaacatcgagtccataattgaatttagtcttacaatgttacagaccttcaaattgtttccataaatctttgaaatatgtcataacacaaaatctgagagtggtttataattttattagataagactcgtccaaatatgctggtgtgagaaatgttgtgatgatataaaaattctaaatagtaatcggtatttttcaaaagaatatttcaattatatacattccatataccatgcctttactaattatatataatacgggatttctcaaagtataaaagaaagataagacaaggaaaacatacaacatctataaaagacgcaccagacagagcctctcattgaattttgtggtaaacgaatagaactctgtaattgagaaaaaaataaaatgaaataatattaagaaataaaaagagagacatatttaagttacaaaattagaaaatattaaatcacgtattaatatttttctaaaacaaaatagggaacttttacttctgttttgaatatataaacgatttttaatagaatttgttcaatattttaatatagtaaataaaaaatttgtatatatgaactattttttttacttgtgtatatatccgatggggttatgaaatttttctatattttgttttttaaaaatatagggttttgtgcaaaacacccatctaactcatctagttttgcaattttaccctatatctctaacaaacacatattaatcttatgaagtctaaaataatacgcaatatgacccttatacgtatattgtaaacttttttacactcccatagattagatttttaatatttcgaaggtcgatttgaacttttcgtcgagaaagcttcgattaaacatattaaatatcaatatcttacaaaatattgtcgtagaaaaatgacgaaaaggggttaaattgcgtattattttagacttcatcggataattatgcattttttggaggtggagaataaagatgcaaaaatagatgagttagagggttttattttgcacaaaaccctaaaaataacaaaatttgatttgataagggtggatatttcactaataaatttgacacgtgtcatgatcacgtgagttaacaattttgaaagcccaactttatgtagatatttggcacatatattggattatgttcattgttttcatgccagatccgaactgctcttcccaaccgtttaacccgctatccagaaactcttctcctctaagattagtgttaacaagtcatggagaacctcattgtatagaagttttacaaactaaacctttagattgattcaaataatttcacgaagaaaatattataaatcgatttatttgtttaaatttggagatataaaccgatgattgattaattatgtgaagccattttacttttattttctctttgtagtcaatatttcttagttaatgttacgtgtttcacacatggagtttcaactatattaaatttctaaaacggctaatcaaatacctaattaacgtattcaaagatgttaaattaaaaaaaattaatatgacagtttcacatttcaatccatataaataagcaaggattttattgttattcatcaatcaaaaacaaacgaagcattaagcatatagttgctatactaagactttttactcaatttctcattatgtctatcaaaaatgtgttttcacttctagcggttctatgtatca

SEQ ID NO: 8 - protein, 1922 aa, Artificial Sequence
  NtPR1a protein sequence
AGAGTTGCCGGTGCAACTCCTCCCATACCAAAATAGTGTTTTATCTTATTTTTAAAATAGATAACTTTTTCAACTTATTTGTTTAAAATTTTAGCTTACGTTACATGTTATAATAATAATTAAAAAAAGAAGAAGATATAGTAATAACATTTGAGAAGTTGCATTTGGGTTCCATGTTTATACATTCTTCACTTCTAATGAATAACACCTTGAAACATCGAGTCCATAATTGAATTTAGTCTTACAATGTTACAGACCTTCAAATTGTTTCCATAAATCTTTGAAATATGTCATAACACAAAATCTGAGAGTGGTTTATAATTTTATTAGATAAGACTCGTCCAAATATGCTGGTGTGAGAAATGTTGTGATGATATAAAAATTCTAAATAGTAATCGGTATTTTTCAAAAGAATATTTCAATTATATACATTCCATATACCATGCCTTTACTAATTATATATAATACGGGATTTCTCAAAGTATAAAAGAAAGATAAGACAAGGAAAACATACAACATCTATAAAAGACGCACCAGACAGAGCCTCTCATTGAATTTTGTGGTAAACGAATAGAACTCTGTAATTGAGAAAAAAATAAAATGAAATAATATTAAGAAATAAAAAGAGAGACATATTTAAGTTACAAAATTAGAAAATATTAAATCACGTATTAATATTTTTCTAAAACAAAATAGGGAACTTTTACTTCTGTTTTGAATATATAAACGATTTTTAATAGAATTTGTTCAATATTTTAATATAGTAAATAAAAAATTTGTATATATGAACTATTTTTTTTACTTGTGTATATATCCGATGGGGTTATGAAATTTTTCTATATTTTGTTTTTTAAAAATATAGGGTTTTGTGCAAAACACCCATCTAACTCATCTAGTTTTGCAATTTTACCCTATATCTCTAACAAACACATATTAATCTTATGAAGTCTAAAATAATACGCAATATGACCCTTATACGTATATTGTAAACTTTTTTACACTCCCATAGATTAGATTTTTAATATTTCGAAGGTCGATTTGAACTTTTCGTCGAGAAAGCTTCGATTAAACATATTAAATATCAATATCTTACAAAATATTGTCGTAGAAAAATGACGAAAAGGGGTTAAATTGCGTATTATTTTAGACTTCATCGGATAATTATGCATTTTTTGGAGGTGGAGAATAAAGATGCAAAAATAGATGAGTTAGAGGGTTTTATTTTGCACAAAACCCTAAAAATAACAAAATTTGATTTGATAAGGGTGGATATTTCACTAATAAATTTGACACGTGTCATGATCACGTGAGTTAACAATTTTGAAAGCCCAACTTTATGTAGATATTTGGCACATATATTGGATTATGTTCATTGTTTTCATGCCAGATCCGAACTGCTCTTCCCAACCGTTTAACCCGCTATCCAGAAACTCTTCTCCTCTAAGATTAGTGTTAACAAGTCATGGAGAACCTCATTGTATAGAAGTTTTACAAACTAAACCTTTAGATTGATTCAAATAATTTCACGAAGAAAATATTATAAATCGATTTATTTGTTTAAATTTGGAGATATAAACCGATGATTGATTAATTATGTGAAGCCATTTTACTTTTATTTTCTCTTTGTAGTCAATATTTCTTAGTTAATGTTACGTGTTTCACACATGGAGTTTCAACTATATTAAATTTCTAAAACGGCTAATCAAATACCTAATTAACGTATTCAAAGATGTTAAATTAAAAAAAATTAATATGACAGTTTCACATTTCAATCCATATAAATAAGCAAGGATTTTATTGTTATTCATCAATCAAAAACAAACGAAGCATTAAGCATATAGTTGCTATACTAAGACTTTTTACTCAATTTCTCATTATGTCTATCAAAAATGTGTTTTCACTTCTAGCGGTTCTATGTATCA

SEQ ID NO: 9 - protein, 197 aa, Artificial Sequence
  NbPINII protein sequence
MAVHKVSFLAHLLVLGIFLLLVDAKACTKECGNFAYGRCPRSQGTPDDPICTSCCAGYKGCNYYNANGTFICEGLSDPRKPNFCPLFCDPDIAYSKCPRSEGETIINPTGCTTCCTGYKGCYYFGQDGKFVCEGESDEPKSCTTECDPRVAYISCPISGLVKINQECINCCNADKGCELYDNDGSLICTGGEPQSAA

SEQ ID NO: 10 - protein, 208 aa, Artificial Sequence
  NbWIN1 protein sequence
MGKLSTVLLALILFIIAAGANAQQCGRQRGGALCGGNLCCSQFGWCGSTPEYCSPSQGCQSQCSGGGGGGGGGGGSAQNVRATYHIYNPQNVGWDLYAVSAYCSTWDGNKPLAWRRKYGWTAFCGPVGPRGRDSCGKCLRVTNTGTGAQTTVRIVDQCSNGGLDLDVNVFRQLDTDGRGNQRGHLIVNYEFVNCGDNMNVLVSPVDKE

SEQ ID NO: 11 - protein, 79 aa, Artificial Sequence
  NbPDF1.2 protein sequence
MAKSQIQCAVFLALFFCFLVASTEMMQKAEALGCEKPSTTWSGPCLFSDDCNNQCINWEGAVHGECSFALGPECFCYFC

SEQ ID NO: 12 - DNA, 3937 bp, Artificial Sequence
  Expression vector with StPINII-NtPR1a - AtMAR1a DNA sequence
caacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtgtatttttacaacaattaccaacaacaacaaacaacaaacaacattacaattactatttacaatggcgcgccccctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattattgaaacgacctacgtccatttcacgttaatatgtatggattgttctgcttgatatcaagaacttaaataattgctctaaaaagcaacttaaagtcaagtatatagtaatagtactatatttgtaatcctctgaagtggatctataaaaagaccaagtggtcataattaaggggaaaaatatgagttgatgatcagcttgatgtatgatctgatattattatgaacacttttgtactcatacgaatcatgtgttgatggtctagctacttgcgatattacgagcaaaattcttaactacatgccttaggaacaagcttacacagttcatataatctactagagggccaaaaacatgaaaattaccaatttagatggtaggaggatattgaaagtggagcagctagttttaataactgaccgttagtcttaaaattgacggtataaaaatatttacataatcaggtcatttataaggtaattataggtaaatatttatgacgaattctcaatagtaatctgaaaaaaaattgtaactaacctattatactaaaactactataataggttagattacattaatcatgtcattagaagatcttagctcggtacccggggatcctctagagtcgacctgcaggcatgcagagttgccggtgcaactcctcccataccaaaatagtgttttatcttatttttaaaatagataactttttcaacttatttgtttaaaattttagcttacgttacatgttataataataattaaaaaaagaagaagatatagtaataacatttgagaagttgcatttgggttccatgtttatacattcttcacttctaatgaataacaccttgaaacatcgagtccataattgaatttagtcttacaatgttacagaccttcaaattgtttccataaatctttgaaatatgtcataacacaaaatctgagagtggtttataattttattagataagactcgtccaaatatgctggtgtgagaaatgttgtgatgatataaaaattctaaatagtaatcggtatttttcaaaagaatatttcaattatatacattccatataccatgcctttactaattatatataatacgggatttctcaaagtataaaagaaagataagacaaggaaaacatacaacatctataaaagacgcaccagacagagcctctcattgaattttgtggtaaacgaatagaactctgtaattgagaaaaaaataaaatgaaataatattaagaaataaaaagagagacatatttaagttacaaaattagaaaatattaaatcacgtattaatatttttctaaaacaaaatagggaacttttacttctgttttgaatatataaacgatttttaatagaatttgttcaatattttaatatagtaaataaaaaatttgtatatatgaactattttttttacttgtgtatatatccgatggggttatgaaatttttctatattttgttttttaaaaatatagggttttgtgcaaaacacccatctaactcatctagttttgcaattttaccctatatctctaacaaacacatattaatcttatgaagtctaaaataatacgcaatatgacccttatacgtatattgtaaacttttttacactcccatagattagatttttaatatttcgaaggtcgatttgaacttttcgtcgagaaagcttcgattaaacatattaaatatcaatatcttacaaaatattgtcgtagaaaaatgacgaaaaggggttaaattgcgtattattttagacttcatcggataattatgcattttttggaggtggagaataaagatgcaaaaatagatgagttagagggttttattttgcacaaaaccctaaaaataacaaaatttgatttgataagggtggatatttcactaataaatttgacacgtgtcatgatcacgtgagttaacaattttgaaagcccaactttatgtagatatttggcacatatattggattatgttcattgttttcatgccagatccgaactgctcttcccaaccgtttaacccgctatccagaaactcttctcctctaagattagtgttaacaagtcatggagaacctcattgtatagaagttttacaaactaaacctttagattgattcaaataatttcacgaagaaaatattataaatcgatttatttgtttaaatttggagatataaaccgatgattgattaattatgtgaagccattttacttttattttctctttgtagtcaatatttcttagttaatgttacgtgtttcacacatggagtttcaactatattaaatttctaaaacggctaatcaaatacctaattaacgtattcaaagatgttaaattaaaaaaaattaatatgacagtttcacatttcaatccatataaataagcaaggattttattgttattcatcaatcaaaaacaaacgaagcattaagcatatagttgctatactaagactttttactcaatttctcattatgtctatcaaaaatgtgttttcacttctagcggttctatgtatca

SEQ ID NO: 13 - DNA, 39 bp, Artificial Sequence
  NbPINII Forward Primer
tggacgagctgtacaagtaattatttctaataagtgatc

SEQ ID NO: 14 - DNA, 39 bp, Artificial Sequence
  NbPINII Reverse Primer
ggatccccgggtaccgagcttcctttcttttcttctttg

SEQ ID NO: 15 - DNA, 38 bp, Artificial Sequence
  NbWIN1 Forward Primer
ggatccccgggtaccgagctagttgacagttaagttaa

SEQ ID NO: 16 - DNA, 39 bp, Artificial Sequence
  NbWIN1 Reverse Primer
tggacgagctgtacaagtaagaagctatatacatatata

SEQ ID NO: 17 - DNA, 39 bp, Artificial Sequence
  NbPDF1.2 Forward Primer
tggacgagctgtacaagtaataattgaaattggattgat

SEQ ID NO: 18 - DNA, 39 bp, Artificial Sequence
  NbPDF1.2 Reverse Primer
ggatccccgggtaccgagcttgctagacttagatgtcaa

SEQ ID NO: 19 - DNA, 36 bp, Artificial Sequence
  AtMA1a at the 5 Forward Primer
cagctatgaccatgattacagagttgccggtgcaac

SEQ ID NO: 20 - DNA, 38 bp, Artificial Sequence
  AtMA1a at the 5 Reverse Primer
gctccaccatgttgagctctgatacatagaaccgctag

SEQ ID NO: 21 - DNA, 37 bp, Artificial Sequence
  AtMA1a at the 3 Forward Primer
gagtcgacctgcaggcatgcagagttgccggtgcaac

SEQ ID NO: 22 - DNA, 39 bp, Artificial Sequence
  AtMA1a at the 3 Reverse Primer
gtaaaacgacggccagtgcctgatacatagaaccgctag

(19) United States
(12) Patent Application Publication
(10) Pub. No.: US 2025/0340896 A1
(43) Pub. Date: Nov. 6, 2025

(54) RECOMBINANT PROTEIN PRODUCTION

(71) Applicant: KBIO HOLDINGS LIMITED, London (GB)

(72) Inventors: Nicholas John HOLTON, Norfolk (GB); AIbor Dobon ALONSO, Norfolk (GB)

(73) Assignee: KBIO HOLDINGS LIMITED, London (GB)

(21) Appl. No.: 18/564,428

(22) PCT Filed: May 26, 2022
(86) PCT No.: PCT/GB2022/051350

§ 371 Date: Nov. 27, 2023

(30) Foreign Application Priority Data
May 27, 2021 (GB) 2107598.1

(51) Int. Cl. C12N 15/82

(52) U.S. Cl. CPC C12N 15/8251; C12N 15/822

(57) ABSTRACT

Disclosed are regulatory elements, expression vectors comprising said regulatory elements for enhancing protein production in plant-based expression systems, and methods for their production and use.

The present application contains a Sequence Listing that has been submitted electronically and is hereby incorporated by reference in its entirety. The electronic Sequence Listing is named Sequence_Listing_ST25.txt, was created on May 16, 2024, and is 37,095 bytes in size.

### FIELD OF THE INVENTION

This invention relates to regulatory elements expression vectors comprising said regulatory elements for enhancing protein production using a plant-based expression system, and their methods of production and use.

### BACKGROUND OF THE INVENTION

Recombinant protein expression in plants can be achieved by generating stable transformed lines or transient expression, in both whole plants and cell cultures. The yield of protein production is critical to commercial viability of the expression platform. Several systems have been developed to achieve high yielding protein production, such as pEAQ plasmid (Sainsbury et al., 2009) (Sainsbury Frank and Lomonossoff George Peter, 2010) and magnICON vector (Gleba et al., 2005). All systems rely on several core features, including a strong promoter and transcription terminator. Additional features are present in some systems to provide viral replication and suppression of silencing.

One of the ways to enhance protein production is to develop an expression system that comprises a terminator region. This is consistent with the eukaryotic systems which comprise a terminator region, located 3′ of the open reading frame (ORF). The terminator regions play multiple roles in the transcription and translation process resulting hence the level of protein production varies significantly between different terminators. The sequence elements that determine the efficiency of terminators is poorly understood. One of the most commonly used terminator in plant expression vectors is the terminator from Agrobacterium tumefaciens T-DNA nopaline synthase (Tnos) which is associated with increased protein production (Depicker et al., 1982). Other plant terminators such as Solanum tuberosum PINII (An et al., 1989), Arabidopsis thaliana HSP18.2 (Nagaya et al., 2010), Nicotiana tabacum extensin (Rosenthal et al., 2018), N. benthamiana ACT3 (Diamos and Mason, 2018) have been identified that result in improved protein production compared to the Tnos terminator.

Another means to enhance protein production, is the addition of tandem terminators (Beyene et al., 2011; Luo and Chen, 2007). In plants, the enhancement in protein production has been observed in both stably transformants and during transient expression. Several combinations of tandem terminators have been investigated, for example, the combination of the cowpea mosaic virus RNA-2 terminator with Tnos (Sainsbury et al., 2009) in the pEAQ expression system and the combination of an intronless extensin and ACT3 which results in the highest protein production reported thus far (Diamos and Mason, 2018).

Enhanced expression and reduced gene silencing have also been observed in expression vectors containing matrix attachment regions (MAR, also known as scaffold attachment regions). The influence on expression has been reported in both stable transformants and transient expression in plants. MAR can be enhanced or repressed or have no influence on gene expression, depending on the host, type of MAR and expression cassette (Dolgova and Dolgov, 2019). The N. tabacum RB7 MAR is most commonly used to enhance gene expression and has been widely used in a number of expression systems (Hall et al., 1991).

Expression of recombinant proteins in plants provides an alternative cost-effective platform for the large-scale production of recombinant proteins. However, plant based-recombinant expression systems offer a lower yield of protein production compared to mammalian expression systems. In light of these developments, it is apparent that there is still a need to identify means to improve plant-based expression system in order to enhance the protein production.

### SUMMARY OF THE INVENTION

The present invention relates to a number of novel terminators from plant stress induced genes, novel MAR derived from Arabidopsis thaliana ECA1 intergenic region and combinations thereof, which are utilised to form vectors to enhance protein expression of a gene of interest in plants.

In accordance with a first aspect, the invention provides a nucleic acid sequence terminator from a stress induced plant gene or fragments or variants of said sequence. The terminator being located in nature 3′ to the open reading frame of the stress induced gene product (“terminator of the invention”). In a further embodiment, the terminators are not a terminator of the following genes: Solanum tuberosum (Sequence ID No 5), Arabidopsis thaliana HSP18.2 and Nicotiana tabacum extensin. In one embodiment, the stress induced plant genes are derived from Nicotiana species eg N. benthamiana or N. tabacum.

In one embodiment, the terminator is a sequence of sequence ID No 1 to 4, a terminator homologue, or a fragment of said sequence, or homologue, or a variant having 80% sequence identity to said sequence ID No 1 to 4, terminator homologue or fragment. The terminator homologue is a terminator of a homologous gene wherein the homologous gene product has at least 30%, 40%, 50%, 60%, 70%, 80% or 90% amino acid sequence identity to sequence ID No 8, 9, 10 or 11. In a preferred embodiment, the homologous gene products have more than 50% sequence identity. In other embodiments, the homologues are identified as having a pairwise comparison E-value of less than 1e-10 returned by BLASTN of a suitable plant nucleotide database, such as Genbank (Sayers et al 2021 [DOI: 10.1093/nar/gkaa1023]).

In some aspects, the present invention provides a nucleic acid of sequence ID No 7 comprising a matrix attachment region or a fragment of at least 300, 400, 500, 600, 700, 800, 900, 1000 base pairs or variant having 70% identity to said region or fragment (“matrix attachment region of the invention”).

In other aspects, the present invention provides an expression vector comprising a sequence of sequence ID No 1 to 4, a terminator homologue, or a fragment of said sequence, or homologue, or a variant having 80% sequence identity to said sequence ID No 1 to 4, terminator homologue or fragment. In other embodiments, the expression vector comprises a nucleic acid of sequence ID No 7 or a variant having 70% identity thereto. In one embodiment, the expression vector comprises a second terminator sequence, wherein the expression vector comprises at least the terminator having a sequence selected from sequence ID 1 to 4. In some embodiments, the expression vector comprises two terminator sequences selected from sequence ID no 1 to 6, homologues, variants, and fragments of said sequence ID No 1 to 6, and terminators having 70, 80, 90% identity to said sequence ID No 1 to 6, homologues, variants and fragments. The two terminators are referred to as tandem terminators.

In other aspects, the present invention provides an expression vector comprising a terminator of the invention, or tandem terminators of the invention and a matrix attachment region of the invention. Suitable terminators are those that comprise a nucleic acid sequence located 3′ to the open reading frame of a stress induced plant gene or fragments or variants of said terminators. Terminators for use in the vectors of the invention comprising a nucleic acid from a stress induced plant gene are those provided in sequence ID No 1 to 4, a terminator homologue thereof, or a fragment of said sequence or said homologue or variant having 80% sequence identity to any of the sequences No 1 to 4 or terminator homologue thereof. In a further embodiment, terminators for use in the vectors of the invention are selected from sequence ID No 1 to 6. The nucleic acid are typically derived from a Nicotiana plant such as N. benthamiana or N. tabacum.

In one embodiment, the matrix attachment region is derived from sequence ID No 7, or it can be a fragment of at least 300 bp or variant having 70% identity to said region or fragment. In some embodiments, the MAR is identified as the ECA1 intergenic region between At5g36340 and At5g36350 from Arabidopsis thaliana. In other embodiments, the expression vector comprises a terminator and two matrix attachment regions. The two matrix attachment regions are selected from sequence ID No 7 or a fragment of at least 300 base pair of sequence ID No 7 or variant having 70% identity to said region or fragment.

In one aspect, the present invention provides an expression vector comprising a promoter, a polypeptide encoding open reading frame, a terminator of sequence ID No 6, a terminator of sequence of sequence ID No 1, and a matrix attachment region of sequence ID No 7.

The expression vectors in accordance with the present invention is used to transform a plant or plant cell in order to express a desired protein in said plant or plant cell. In one embodiment, the expression of protein is transient. The nucleic acid of the stress induced terminators of sequence ID 1 to 6 may be used in the production of stable plants or plant cells, or may be used to transform a plant to express a protein of interest, wherein the expression is transient.

### DETAILED DESCRIPTION OF THE INVENTION

As used herein a “terminator” is a DNA sequence that causes the dissociation of RNA polymerase from DNA and thus terminates the transcription of DNA into messenger RNA. The “terminator” contains DNA sequences that permit site-specific cleavage of the RNA transcript and allow the formation of polyA tail at the 3′ end of the transcript. The “terminator” also contains sequences that bind proteins that promote the protein translation of the messenger RNA.

As used herein the term “homologue” when used in the context of a terminator refers to the terminator of a homologous gene which homologous genes, are genes related to a second gene by descent from a common ancestral DNA sequence. The term, homolog, may apply to the relationship between genes separated by the event of speciation (ortholog) or to the relationship between genes separated by the event of genetic duplication (paralog). The terminator of the homologous genes is located in the 1000 base sequence immediately following the stop codon and prior to the start of any open reading frame of a second gene.

As used herein a “variant” is a nucleic acid sequence having 70, 80, 90, 95, 99% identity over the entire length of the reference sequence whilst retaining the biological activity of the reference sequence.

As used herein sequence identity is preferably determined by the number of identical nucleotides or amino acids in the same position in aligned sequences. Amino acid percent identity is determined using the BLASTP alignment using the BLOSUM62 substitution matrix, a gap existence penalty of 11, and a gap extension penalty of 1. DNA percent identity is determined using the BLASTN alignment used match score of 1, mismatch score of −2, a word size of 7, a gap existence penalty of 5, and a gap extension penalty of 2.

A “fragment” as used herein is a portion of a nucleic acid that retains the biological function of a reference sequence.

As used herein a “matrix attachment region” or scaffold attachment region is a sequence in the DNA of eukaryotic chromosomes where the nuclear matrix attaches. In the context of the present invention these regions are derived from the ECA1 intergenic region between At5g36340 and At5g36350 from Arabidopsis thaliana and mediate structural organization of the chromatin within the nucleus and promote gene expression. MARS can be identified by binding to the nuclear matrix or predicted by the presence of AT-rich DNA (>70%). MARS can also be predicted using computer algorithms, such as SMARTest (Frisch et al 2002 [doi: 10.1101/gr.206602]) or MAR-finder (Singh et al 1997 [doi: 10.1093/nar/25.7.1419]).

The term “transient expression” refers the temporary expression of genes to produce a protein of interest that are expressed for a short time, up to 4 weeks, after ahttps://en.wikipedia.org/wiki/Nucleic_acid nucleic acid, most frequently plasmid DNA or plasmid fragment encoding an expression cassette, has been introduced into plant cells and contrast with stable expression which enables expression of the protein over many generations.

“An expression vector” as used herein is ahttps://en.wikipedia.org/wiki/Plasmid plasmid or virus designed for gene expression in cells. The expression vector is used to introduce a specific gene of interest into a target cell, and can commandeer the cell's mechanism to produce the protein encoded by the gene. An expression vector as used herein comprises at least one expression cassette.

As used herein an “expression cassette” refers to a component of the expression vector which contains the gene of interest under the control of the regulatory sequences for expression. An expression cassette as described herein comprises a promoter sequence, a 5′UTR (5′ untranslated region) such as tobacco etch virus omega 5′UTR, an open reading frame, and a terminator. CaMV 35S is a promoter derived from the cauliflower mosaic virus. Other suitable promoters include Cassava vein mosaic virus promoter, promoter of the small subunit of ribulose biphosphate carboxylase, plastocyanin promoter, ubiquitin promoter from monocots and dicots, and the actin promoter. Promoters may be constitutive, inducible or tissue specific.

As used herein “Viridiplantae” are plants including but not limited to Nicotiana species (such as, tobacco (N. tabacum) and N. benthamiana and others), carrot, legumes (such as, cowpea, alfalfa/lucerne, beans, chickpea, peanut, pea), papaya, potato, rapeseed/canola, apple, vegetable brassicas (such as beets, cabbages, cauliflower, broccoli, and others), rice, maize, soybean, tomato, grapes, rose, carnation, citrus(including lemons/limes, oranges, grapefruits, tangerines, and others), sorghum, sugarcane, barley, banana, cassava, cane berries (rubus), coffee, curcurbits (including cucumbers, melon, squashes, watermelon, and others), hazelnut, hop, lettuce, okra, olive, peanut, rye, strawberry, sweet potato, pear, cyclamen, impatiens, kalanchoe, geranium, gerbera, cattelya, chrysanthemum, cineraria, fennel, seed sprouts, avocado, kiwi, oats, pea, stone fruits (such as almond, plum, apricot, cherry, peach/nectarine and others), passion fruit, pepper, celery, tobacco, wheat, onion/garlic, and cannabis. These may also include plant cell suspensions of the species described above. Preferably, the host is selected from: Nicotiana species (such as N. tabacum and N. benthamiana), potato, brassicas, rice, maize, soybean, tomato, lettuce and wheat.

The expression vectors in accordance with the present invention are used introduced into a plant or plant part using standard methods. The transformation as used in the present invention is widely used in many plant species. Agrobacterium tumefaciens (A. tumefaciens) is a gram negative soil bacterium used as the delivery vector to infect plants.

The expression vector comprises at least one expression cassette. An expression cassette comprises a single stress induced terminator or tandem terminators (also referred to as “double terminator”). A single stress induced terminator is located at the 3′ of the open reading frame of the expression cassette.

“Pairwise comparison E-value” as used herein is the E-value generated when a comparison of two sequences of interest is performed.

“Pfam” in accordance with the present invention is the database Pfam version 34.0 that is based on UniProt release 2020_06 and is a large collection of protein families, each represented by multiple sequence alignments and hidden Markov models (HMMs) (Jaina Mistry, Sara Chuguransky, Lowri Williams, Matloob Qureshi, Gustavo A Salazar, Erik L L Sonnhammer, Silvio C E Tosatto, Lisanna Paladin, Shriya Raj, Lorna J Richardson, Robert D Finn, Alex Bateman, Pfam: The protein families database in 2021, Nucleic Acids Research, Volume 49, Issue D1, 8 Jan. 2021, Pages D412-D419, https://doi.org/10.1093/nar/gkaa913). Proteins are generally composed of one or more functional regions, commonly termed domains. Different combinations of domains give rise to the diverse range of proteins found in nature. The identification of domains that occur within proteins can therefore provide insights into their function.

Stress induced terminators are those whose transcript and/or protein abundance is increased by the application of a biotic or abiotic stress signal to a plant. A significant increase is defined as >2 fold increase in transcript or protein from the uninduced state. Typically, gene induction can be detected 3-5 hours of the stress signal. Biotic stresses, include bacteria, fungi, nematodes, insects and viral organisms or chemicals or proteins derived from these organisms. Biotic stress can also be induced through herbivory or salivary proteins or chemicals. Abiotic stress can be induced by wounding, extreme temperature, salinity, water excess or deficit, light or ultraviolet radiation, and chemical toxins, such as fertilizers and heavy metals. Stress signals regulate gene expression altering the number of transcripts and protein generation. In order to respond to these stresses plants initiate a defence response that often requires the rapid and high level induction of specific proteins involved in the protection of the plant from the stress. This is exemplified by the induction of NtPR1a in response to pathogen infection, where gene induction begins within 3 hours of pathogen recognition and results in the high-level accumulation of NtPR1a protein.

In accordance with the present invention, stress induced terminators are, in a preferred embodiment, derived from stress induced genes such as the N. tabacum pathogenesis-related protein 1a (referred to as NtPR1a), N. benthamiana proteinase inhibitor type-2 (referred to as NbPINII), wound-induced protein (referred to as NbWIN1), and defensin-like protein 14 (referred to as NbPDF1.2). The terminators of these stress induced genes result in enhanced expression of heterologous recombinant protein.

Terminators that improve protein production already known to a person skilled in the art include the terminator from Agrobacterium tumefaciens T-DNA nopaline synthase (Tnos). This is a commonly used terminator in plant expression vectors (Depicker et al., 1982). Other terminators that have been identified include Solanum tuberosum PINII (An et al., 1989), Arabidopsis thaliana HSP18.2 (Nagaya et al., 2010), Nicotiana tabacum extensin (Rosenthal et al., 2018), N. benthamiana ACT3 (Diamos and Mason, 2018).

Novel stress induced terminators identified by the present invention are N. tabacum pathogenesis-related protein 1a (Gen bank ID: X12737.1)(referred to as NtPR1a) (SEQ ID No: 1) terminator, N. benthamiana proteinase inhibitor type-2 (Niben101Scf07757g00001.1)(referred to as NbPINII) (SEQ ID No:2) terminator, wound-induced protein (Niben101Scf01015g01002.1) (referred to as NbWIN1) (SEQ ID No:3) terminator, and defensin-like protein 14 (Niben101Scf00761g00006.1)(referred to as NbPDF1.2)(SEQ ID No:4) terminator. In each case the terminator sequence is located 3′ to the stop codon of the open reading frame.

Terminators as described above can be used in accordance with the present invention as part of a tandem terminator combination and additionally StPINII (SEQ ID NO: 5) terminator, Tnos (SEQ ID NO: 6) terminator may also be combined with the novel stress induced terminators of the invention.

To exemplify still further the following is a list of stress induced terminators provided by the invention:

### NtPR1a

Plant terminators of genes containing a protein with >70% Identity to NtPR1a (Sequence ID No: 8) and or contain homology to Pfam domain PF00188 (cysteine-rich secretory protein superfamily) with an E-value of <0.01.

### NbPINII

Plant terminators of genes containing a protein with >70% Identity to NbPINII (Sequence ID No: 9) and or contain homology to Pfam domain PF02428 (Potato type II proteinase inhibitor family) with an E-value of <0.01.

### NbWIN1

Plant terminators of genes containing a protein with >70% Identity to NbWIN1 (Sequence No: 10) and or contain homology to Pfam domain PF00967 (Barwin) and PF00187 (Chitin binding 1) with an E-value of <0.01.

### NbPDF1.2

Plant terminators of genes containing a protein with >70% Identity to NbPDF1.2 (Sequence ID No: 11) and or contain homology to Pfam domain PF00304 (Gamma-thionin family) with an E-value of <0.01.

E-value as mentioned above is a parameter the statistical significance of a match. The lower the E-value the more significant the match is. It is preferable in accordance with the present invention to identify other terminators with a pairwise comparison E-value of less than 1e-10.

Tandem terminators are two terminators that have been fused together. The two terminators are either fused together without a linker sequence, or the two terminators are linked via a linker sequence. In one embodiment, a linker sequence may encompass up to 200 base pairs. Tandem terminators may be two of the same terminators fused together such as StPINII+StPINII, or it may be a combination of two different terminators such as StPINII+NtPR1a. Tandem terminators are both located at the 3′ of the open reading frame of the expression cassette. The second terminator is joined at the 3′ of the first terminator. Any of the following terminators: S. tuberosum PINII, N. benthamiana PINII, WIN1, PDF1.2 and N. tabacum PR1a can be fused with any of the following: Tnos, S. tuberosum PINII, N. benthamiana PINII, WIN1, PDF1.2 and N. tabacum PR1a. Preferably tandem terminators are selected from the following group: NbPDF1.2+Tnos, NbPINII+Tnos, NbWIN1+Tnos, StPINII+EU, StPINII+HSP18, StPINII+Tnos, StPINII+StPINII, StPINII+NtPR1a, NtPR1a+Tnos, NtPR1a+StPINII, and NtPR1a+NtPR1a. The aforementioned pairs of terminators are arranged as listed above, for example NbPDF1.2 is the first terminator arranged upstream of the second terminator Tnos and this arrangement is denoted as “NbPDF1.2+Tnos”. In another embodiment, the pairs may be arranged in a different order. More preferably, the combination of StPINII+NtPR1a is desirable.

The expression cassette further comprises a matrix attachment region (MAR). MARs can be located either at the 5′UTR and/or at the 3′UTR of an expression vector comprising the terminator. In some embodiments, a MAR is upstream of an expression cassette comprising the single or tandem terminator(s). In other embodiments, a MAR is downstream of an expression cassette comprising the single or tandem terminators. In a preferred embodiment, a MAR flanks either side of the expression cassette thereby being upstream and downstream of an expression cassette containing a single or tandem terminator(s). The most commonly used MAR in a number of expression systems is known as the N. tabacum RB7 MAR (Hall et al., 1991). The present invention has identified a novel MAR which is the intergenic region between Arabidopsis thaliana At5g36340 translational start codon and At5g36350 translational stop codon (referred to as AtMAR1a; Genbank ID: CP002688.1 from bases 14331550 to 14333550). In one embodiment, the expression vector comprises a tandem terminator and a MAR which is upstream of the expression cassette. In other embodiments, the expression vector comprises a tandem terminator and two MARs, wherein each MAR flanks either side of the expression cassette, i.e. the first MAR is upstream of the expression cassette and the second MAR is downstream of the expression cassette. In one embodiment, the MAR is AtMAR1a and it is upstream of an expression cassette containing the double terminators, for example StPINII+NtPR1a. In other embodiments, the MAR is AtMAR1a and it is upstream and downstream of an expression cassette containing double terminators, for example StPINII+NtPR1a.

The presence of selected single stress induced terminators or tandem terminators increases protein production from the expression vector. This effect was further enhanced in the presence of the MAR of the invention. Increased protein production is determined by the enhanced expression of GFP, which is determined by the emission of the florescence intensity. The expression vectors as used herein comprises the reporter gene GFP in order to measure the protein production in the presence of single or tandem terminator (s) and/or MAR(s). The increase in protein production is more than 1.3-fold, 5-fold or 10-fold.

p19 is a suppressor of RNA silencing which is derived from tomato bushy stunt virus. The pEAQ-HT vector backbone comprises p19 (Sainsbury, F., Thuenemann, E. C. and Lomonossoff, G. P. (2009), pEAQ: versatile expression vectors for easy and quick transient expression of heterologous proteins in plants. Plant Biotechnology Journal, 7: 682-693. https://doi.org/10.1111/j.1467-7652.2009.00434.x). In certain embodiments of the invention the expression vectors additionally comprise p19 gene. Other suitable silencing suppressors include but are not limited to Pothos latent aureusvirus (P14), Turnip crinkle virus (P38), tomato yellow leaf curl virus (V2), Tomato aspermy cucumovirus (2b), Tobacco etch virus (HC-Pro), Beet yellows virus (P21), poleroviruse (P0) and Sweet potato mild mottle ipomovirus (P1).

Identification of homologous terminators is difficult given that the evolutionary constraints on non-protein coding sequences are significantly lower than protein coding sequences. Therefore, the degree of sequence identity between the promoters and terminators of an orthologous protein coding gene may have a high degree of sequence variation. However, despite this divergence in sequence identity they are still capable of performing the same biological function, such that the induction or tissue specificity of protein expression is maintained by the promoter and terminator sequences. In accordance with the present invention, the homologous terminators are identified using a method that does not rely on the sequence identity of the terminator itself, but rather the identity of the protein that is linked to the terminator. The first step in this methodology is to identify related protein sequences, this can be performed by several sequence homology methods, including but not limited to BLASTP, against a protein database containing plant sequences with the protein sequence of interest. This search will reveal homologous sequences, with a preferred sequence identity of >30%. Homologous sequences can also be identified using defined sequence motifs that are found in the class of protein. Motifs are described in databases such a Pfam version 34.0 that is based on UniProt release 2020_06. Homologous protein sequences can be identified by the presence of these sequence motifs, using standard bioinformatic methods. A target can be selected from this list and the genomic sequence containing the terminator can be identified using standard bioinformatic methods. The terminator sequence is the sequence 3′ of the terminator codon of the gene and this will encompass at least 300-2000 base pairs in length.

### FIGURES

FIG. 1 is a schematic diagram of terminator vector. FIG. 1A illustrates a single terminator vector comprising a 35 Ω-eGFP-terminator expression cassette. FIG. 1B illustrates a tandem terminator vector comprising a 35 Ω-eGFP-terminator expression cassette with two terminators fused to each other. FIG. 1C illustrates an AtMAR1a vector comprising two AtMAR1a flanking either side of the 35 Ω-eGFP-terminator expression cassette.

FIG. 2 shows the intensity of the fluorescence emitted by vectors comprising a single stress induced terminator selected from: StPINII, NtPR1a, NbPINII, NbWIN1, NbPDF1.2 compared to a vector comprising Tnos and pEAQ-HT. Stress-induced terminators resulted in enhanced expression.

FIG. 3 shows the intensity of the fluorescence emitted by vectors comprising combinations of tandem terminators (NbPINII+Tnos, NbPDF1.2+Tnos, and NbWIN1+Tnos) showed enhanced eGFP expression compared to vectors comprising a single stress induced terminator alone (NbPINII, NbPDF1.2 and NbWIN1) and EU-ACT.

FIG. 4 shows the intensity of the fluorescence emitted by vectors comprising a tandem terminator combination of StPINII terminator in combination with StPINII, Tnos, HSP18.2, EU, or NtPR1a. Tandem terminators showed enhanced eGFP expression compared to vectors comprising a single stress induced terminator alone, pEAQ-HT and EU-ACT.

FIG. 5 shows the intensity of the fluorescence emitted by vectors comprising AtMAR1a, vectors without AtMAR1a, vectors comprising Tnos and pEAQ-HT. Vectors with AtMAR1a resulted in increased fluorescence intensity in comparison the vector without AtMAR1a, Tnos and pEAQ-HT.

FIG. 6 shows the intensity of the fluorescence emitted by vectors comprising AtMAR1a flanking either side of the 35SΩ-eGFP-terminator expression cassette comprising two terminators fused to each other, vectors comprising a AtMAR1a at the 3′ of the 35SΩ-eGFP-terminator expression cassette comprising two terminators fused to each other, vectors comprising Tnos and pEAQ-HT. Vectors with AtMAR1a resulted in increased fluorescence intensity in comparison the vector without AtMAR1a, Tnos and pEAQ-HT.

### EXAMPLES

Certain aspects and embodiments of the invention will now be illustrated by way of example and with reference to the figures described above.

### Example 1: Expression Vector Comprising a Single Terminator

### Synthesis of Terminators

N. tabacum pathogenesis-related protein 1a [PR1a] (NtPR1a, X12737.1) terminator was chemically synthesised to yield sequence ID No: 1. N. benthamiana terminators proteinase inhibitor type-2 [PINII] (NbPINII, Niben101Scf07757g00001.1), Wound-induced protein [PR4] (NbWIN1, Niben101Scf01015g01002.1) and defensin-like protein 14 [PDF1.2] (NbPDF1.2, Niben101Scf00761g00006.1) were amplified by PCR from N. benthamiana genomic DNA using the primers listed in Table 1. S. tuberosum PINII (StPINII) terminator was synthesised to yield sequence ID No:5. Tnos was synthesised to yield sequence ID No: 6.

TABLE 1
Primers for amplification of NbPINII,
NbWIN1 and NbPDF1.2

| Gene | Forward primer | Reverse primer |
|---|---|---|
| NbPINII | TGGACGAGCTGTACAAGTAA | GGATCCCCGGGTACCGAGCT |
| | TTATTTCTAATAAGTGATC | TCCTTTCTTTTCTTCTTTG |
| NbWIN1 | GGATCCCCGGGTACCGAGCT | TGGACGAGCTGTACAAGTAA |
| | AGTTGACAGTTAAGTTAA | GAAGCTATATACATATATA |
| NbPDF1.2 | TGGACGAGCTGTACAAGTAA | GGATCCCCGGGTACCGAGCT |
| | TAATTGAAATTGGATTGAT | TGCTAGACTTAGATGTCAA |

### Generation of Single Terminator Expression Vectors

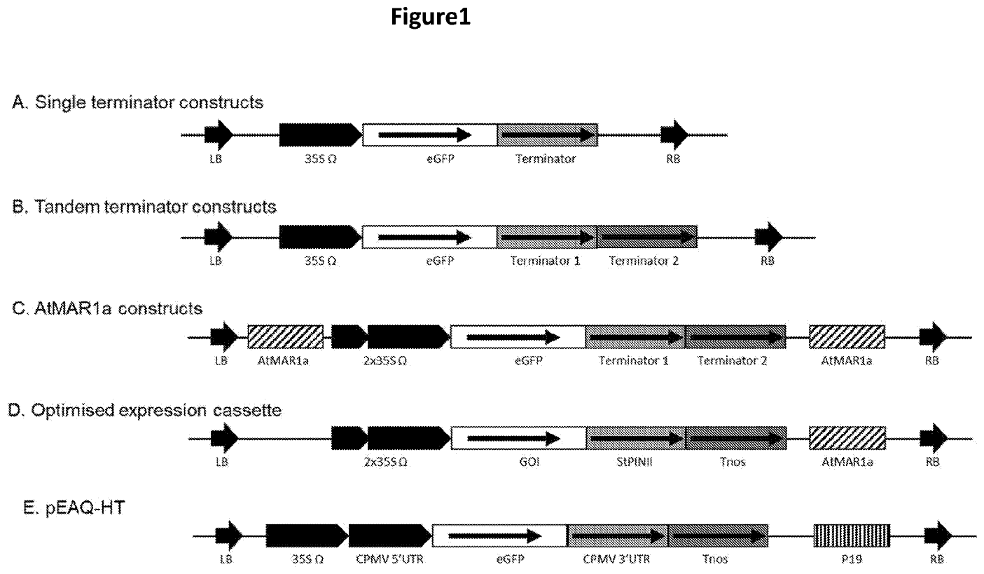

Binary vector plasmids comprising a promoter consisting of a single CaMV 35S promoter with a TEV omega enhancer were used to generate the expression vectors. An eGFP was cloned into the pEAQ-HT to generate 35 Ω-eGFP-terminator expression vector. Single terminators selected from: PR1a, PINII, NbPINII, NbWIN1, PDF1.2, StPINII, and Tnos, were cloned into individual vectors. A schematic diagram of the expression vectors is shown in FIG. 1A.

Expression from a Single Terminator Vector

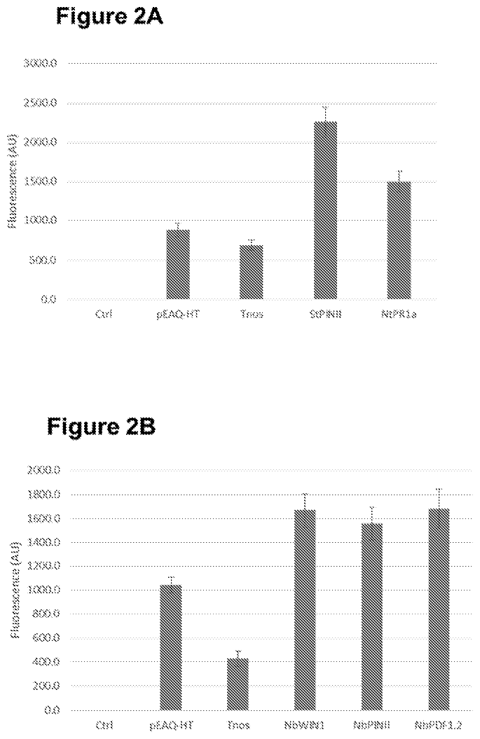

A. tumefaciens containing the binary vectors with the terminator were co-infiltrated with A. tumefaciens carrying the P19 silencing suppressor into N. benthamiana leaves, except for pEAQ-HT vectors where P19 is present on the vector. Plants were grown for six days post infiltration, when samples were taken and eGFP expression monitored by fluorescence measurement using a fluorescence plate reader with an excitation of 488 nm and emission 507 nm. ELISA quantification of eGFP was performed using GFP ELISA kit (Abcam) following the manufactures instructions. At 6 days post infiltration (dpi), eGFP expression was compared between the different terminators. Vectors comprising StPINII, NtPR1a, NbPINII, NbWIN1, and NbPDF1.2 terminators emitted increased fluorescence intensity compared to vector comprising Tnos or pEAQ-HT vector (FIG. 2A and FIG. 2B). Increased fluorescence intensity demonstrates enhanced eGFP expression by at least 1.3 fold and at least by 1.5 fold by vectors comprising one of the following terminators: StPINII, NtPR1a, NbPINII, NbWIN1, and NbPDF1.2, compared to pEAQ-HT and the vector comprising Tnos, respectively—thus demonstrating the expression of a protein is enhanced by the terminators of the invention.

### Example 2: Expression Vector Comprising Tandem Terminators

### Generation of Tandem Terminator Expression Vectors

The expression vectors were generated as described above in Example 1. Tandem terminators were created by fusion of stress induced terminators (NbPDF1.2, NbPINII, NbWIN1) with Tnos, HSP18.2, EU, StPINII or NtPR1a in the second position terminators through NEB HiFi® DNA assembly. These tandem terminators were cloned into a binary vector with the cauliflower mosaic virus (CaMV) 35S promoter, and eGFP, to generate 35SΩ-eGFP-terminator expression cassette. A schematic diagram of the expression vectors is shown in FIG. 1B. eGFP was cloned into pEAQ-HT.

Expression from Vectors Containing Tandem Terminators

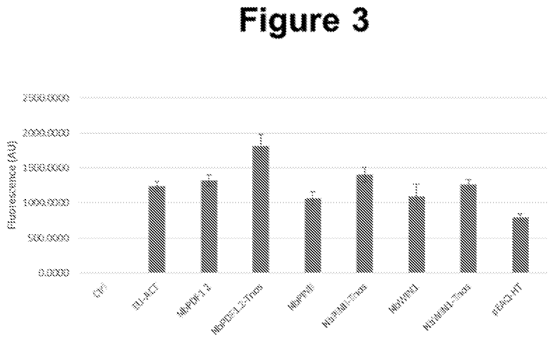

A. tumefaciens vectors where P19 is present on the vector. Plants were grown for six days post infiltration, when samples were taken and eGFP expression monitored by fluorescence measurement using a fluorescence plate reader with an excitation of 488 nm and emission 507 nm. ELISA quantification of eGFP was performed using GFP ELISA kit (Abcam) following the manufactures instructions. Expression of eGFP was compared between vectors comprising a single stress induced terminator and vectors comprising tandem terminators. Different combinations of tandem terminator combinations of stress induced terminators (NbPDF1.2, NbPINII, NbWIN1) with Tnos, HSP18.2, EU, StPINII or NtPR1a in the second position were generated. Expression of eGFP were compared to pEAQ-HT or the state of the art EU-ACT tandem terminator at 6 dpi (FIG. 3).

Vectors comprising tandem terminators (NbPINII+Tnos, NbPDF1.2+Tnos, and NbWIN1+Tnos) showed enhanced eGFP expression compared to vectors comprising a single stress induced terminator alone (NbPINII, NbPDF1.2 and NbWIN1). Expression of eGFP with NbPINII-Tnos, NbPDF1.2-Tnos and NbWIN1-Tnos were equivalent or enhanced compared to EU-ACT. Single and tandem combinations of NbPINII, NbPDF1.2 and NbWIN1 all resulted in significantly higher expression than pEAQ-HT showing the improvement of the vectors of the invention.

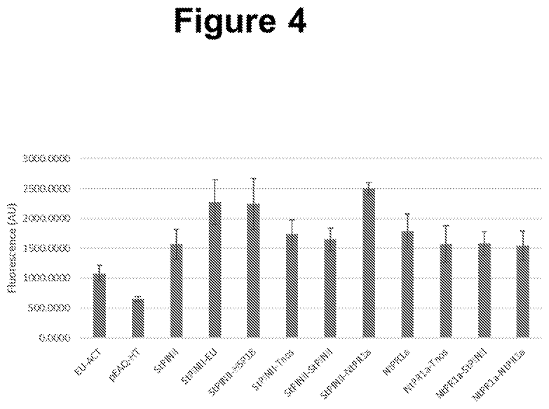

Tandem terminator combinations were generated by cloning the StPINII terminator in combination with StPINII, Tnos, HSP18.2, EU, or NtPR1a in the second position into the vectors. The tandem terminator combinations resulted in increased expression of eGFP compared to the StPINII terminator alone (FIG. 4). No significant enhancement was observed with the addition of a second copy of StPINII or Tnos. StPINII alone resulted in enhanced expression compared to both EU-ACT and pEAQ-HT. Tandem terminator combinations were generated by cloning the NtPR1a terminator in combination with StPINII, Tnos, or NtPR1a in the second position into the vectors. Tandem terminator combinations with NtPR1a did not significantly enhance expression compared to NtPR1a alone (FIG. 4).

### Example 3: Expression Vector Comprising MAR

### Synthesis of MAR

AtMAR1a was amplified by PCR from Arabidopsis thaliana to yield sequence ID No: 6.

### Generation of an Expression Vector Comprising a MAR and a Tandem Terminator

Two AtMAR1a was cloned using two sets of primers listed in Table 2 (primer to clone at 5′ and primer to clone at 3′), flanking either side of an expression cassette containing a promoter consisting of a double enhancer CaMV 35S promoter with a TEV omega enhancer, eGFP and a tandem terminator comprising StPINII and Tnos (2×35SΩ-eGFP-StPINII-Tnos). A schematic diagram of the expression vectors is shown in FIG. 1C. A single AtMAR1a was cloned using a single primer listed in Table 2 (primer to clone at 3′), at the 3′ of an expression cassette containing a promoter consisting of a double enhancer CaMV 35S promoter with a TEV omega enhancer, eGFP and a tandem terminator comprising StPINII and Tnos (2×35SΩ-eGFP-StPINII-Tnos).

TABLE 2
Primers for cloning AtMAR1a

| Gene | Forward primer | Reverse primer |
|---|---|---|
| AtMA | CAGCTATGACCATGATTA | GCTCCACCATGTTGAGCT |
| 1a at | CAGAGTTGCCGGTGCAAC | CTGATACATAGAACCGCT |
| the 5′ | | AG |
| AtMA | GAGTCGACCTGCAGGCAT | GTAAAACGACGGCCAGTG |
| 1a at | GCAGAGTTGCCGGTGCAA | CCTGATACATAGAACCGC |
| the 3′ | C | TAG |

### Expression in a MAR Containing Vector

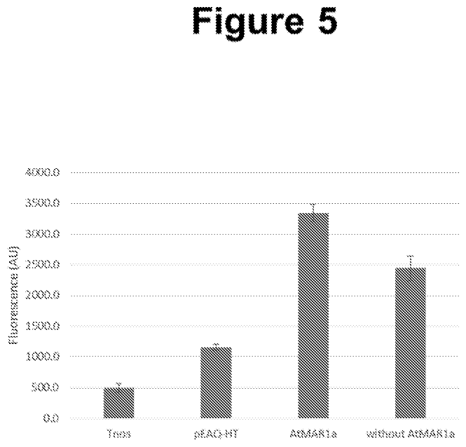

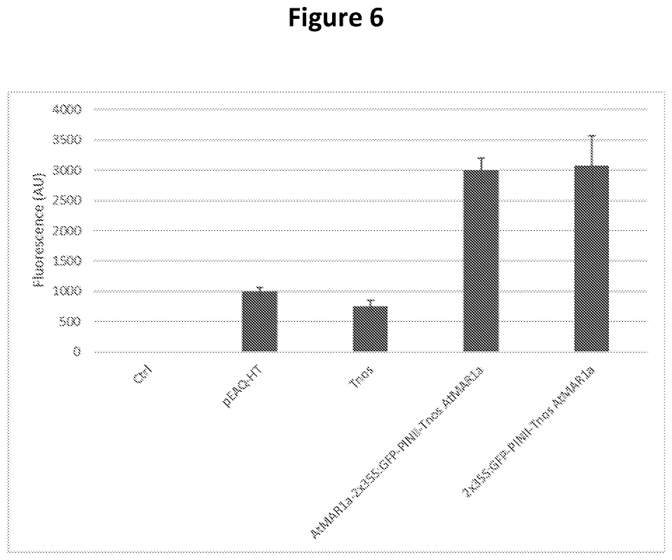

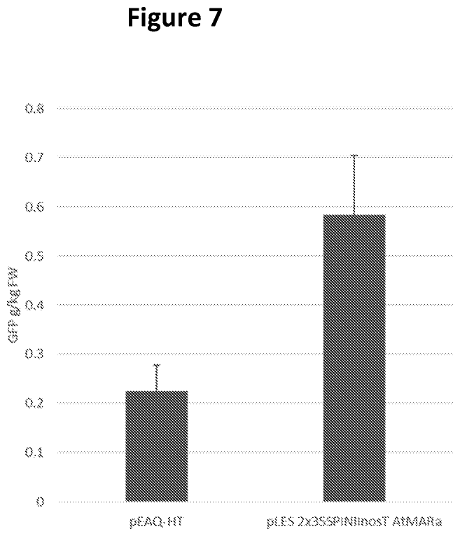

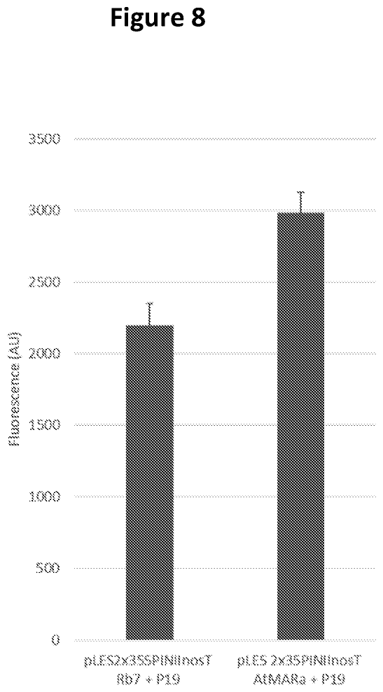

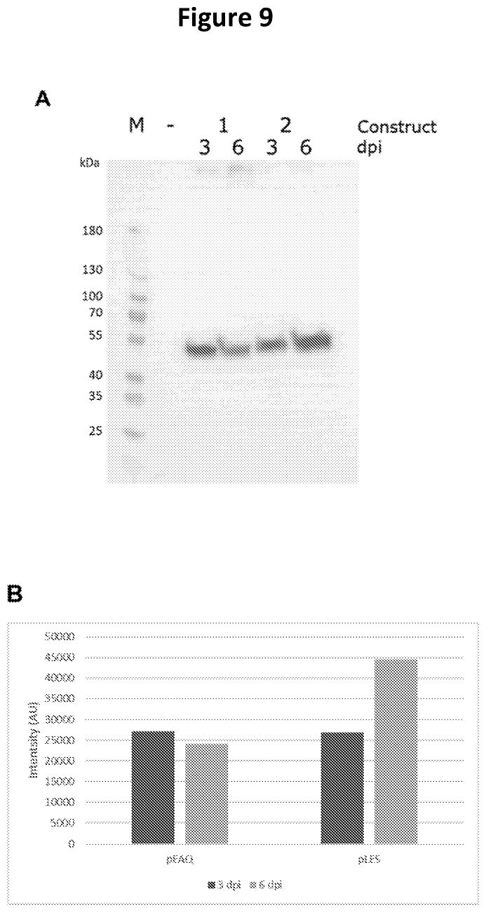

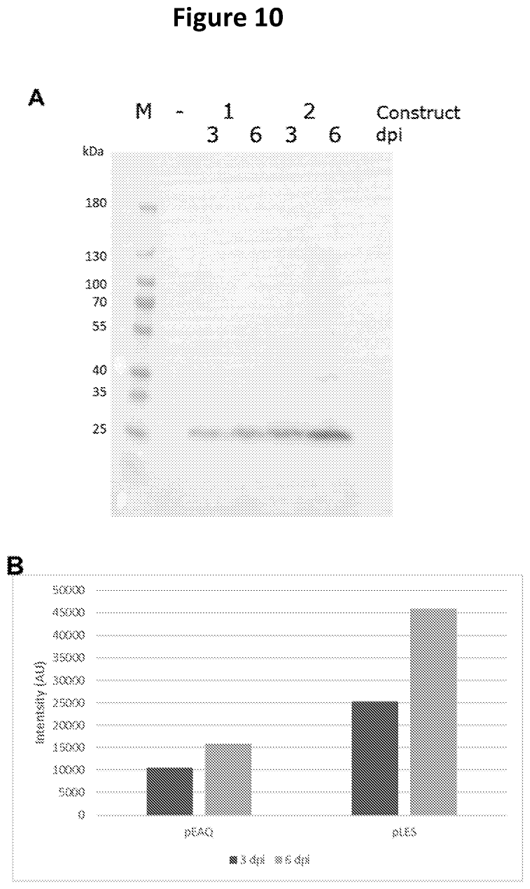

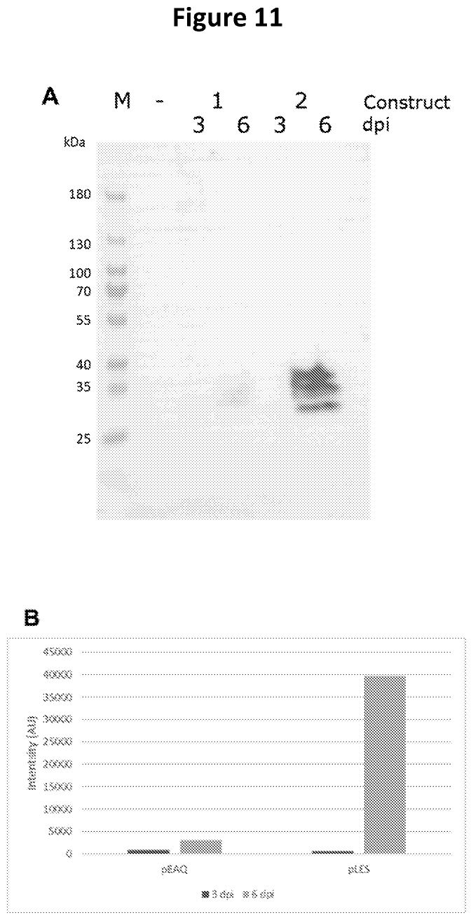

A. tumefaciens containing the binary vectors with the terminator were co-infiltrated with A. tumefaciens carrying the P19 silencing suppressor into N. benthamiana leaves, except for pEAQ-HT vectors where P19 is present on the vector. Plants were grown for six days post infiltration, when samples were taken and eGFP expression monitored by fluorescence measurement using a fluorescence plate reader with an excitation of 488 nm and emission 507 nm. ELISA quantification of eGFP was performed using GFP ELISA kit (Abcam) following the manufactures instructions. Vectors comprising two AtMAR1a or a single AtMAR1a resulted in increased fluorescence intensity in comparison the vector without AtMAR1a, vector comprising the Tnos terminator and pEAQ-HT (FIG. 5 and FIG. 6). The addition of AtMAR1a resulted in greater than 1.3 fold increase in expression and greater than 2.5 fold increase compared to pEAQ-HT (FIG. 5).

### Sequences

| Sequence 1: NtPR1a terminator DNA sequence | |
|---|---|
| >X12737.1 Tobacco PR-1a gene for pathogenesis- | |
| related protein 1a | |
| TTGAAACGACCTACGTCCATTTCACGTTAATATGTATGGATTGTTCTGCTTGATATCAAGAACTTAAATAATTGCTCT | |
| AAAAAGCAACTTAAAGTCAAGTATATAGTAATAGTACTATATTTGTAATCCTCTGAAGTGGATCTATAAAAAGACCAA | |
| GTGGTCATAATTAAGGGGAAAAATATGAGTTGATGATCAGCTTGATGTATGATCTGATATTATTATGAACACTTTTGT | |
| ACTCATACGAATCATGTGTTGATGGTCTAGCTACTTGCGATATTACGAGCAAAATTCTTAACTACATGCCTTAGGAAC | |
| AAGCTTACACAGTTCATATAATCTACTAGAGGGCCAAAAACATGAAAATTACCAATTTAGATGGTAGGAGGATATTGA | |
| AAGTGGAGCAGCTAGTTTTAATAACTGACCGTTAGTCTTAAAATTGACGGTATAAAAATATTTACATAATCAGGTCAT | |
| TTATAAGGTAATTATAGGTAAATATTTATGACGAATTCTCAATAGTAATCTGAAAAAAAATTGTAACTAACCTATTAT | |
| ACTAAAACTACTATAATAGGTTAGATTACATTAATCATGTCATTAGAAGATCTT | |
| Sequence 2: NbPINII terminator DNA sequence | |
| >NbPINII term [Niben101Scf07757g00001.1] | |
| TTATTTCTAATAAGTGATCTGTGATATTATTGTATATGAAATAAAAGCATGATAGATATATATATGCACTGTCATGCT | |
| AAACTCTGTAATGTGGGCAGTGGAGATGTTTTCCACAGCTTGCGCAAGGACATGCCCAAGAATTTAATGAATATTATG | |
| AACCACTAGTTTCTTTTACTTCTGAACTCTATCGTACTAGTAATTGGTTTACTTAATTACTTAACTTTTAATGAAGCT | |
| TTTCCGTTGAAATAGATCAAGTATAAGGGAGAAACTGGACGACCGAAAGAGACCCGATCTCTATGCAATCTCTATCTA | |
| CCTATCTATCTATCCATCTCCATATCTGTCTGTCTATCTTTCTTCTTGGCATCTCCCATAGTCAAAATTACCACACTC | |
| TTTGGTACAAACCTTGGCATCAACAAGTAGTAGAAACATTCCTAAAATAAACATATAGCTATATTTGTTAGTTATTTC | |
| CATGCCTTATACTTTAATCTCATATTCGAATTTTATTCTTTTTTTTTAAGGAATGGTAAACCAAAAATGGCAATAACT | |
| CACACAATTTAAAGGATTACACATGACAATCTTAAGGAAAAACAACATCTCCATATCAAAGAAGAAAAGAAAGGA | |
| Sequence 3: NbWIN1 terminator DNA sequence | |
| >NbPR4 term [Niben101Scf01015g01002.1] | |
| GAAGCTATATACATATATATGGCCATGTTTACCCTTTGACGGCCCAAATAAAAGTAAAAGAACGATATGTAAAAGGAA | |
| AAAGAAAATAAAGTTGCTTTGATTGGGTTATGCAATTCCAATATCTATTCAAGAATTAATGTCTTTCGTTTGGGAAGA | |
| ATGAGGTGAAGTGTGTATTATCTTTGTGATTTTAAATAAAAAAATCACAGTGGGACAGTATTTGTGATTATATTAGGT | |
| TGCTGTTGTTGTATTTTAGTAGTTATTCCTTTTAGTTTTTACCCAACACCCCAATTTGCAAAGGATTGCACAAGCTGA | |
| GTTCACACCTGCTACTTCCAAACACAAAATGTCATCTACATAAGACAACTAATGTATATAAAGCAAAAATAGAACTAC | |
| CATCTTTTGGAAGAGATGTAACTAGAACTAGCTAGGAAATAATAACTTGGAATATCAGAAGAATGTATTCCCTAGGTT | |
| CGATGGATGTTTGTATTATTGCTAGAACATATTACATAGATTATGAATATTAAAATAAGCGCAGGCAGATTTTGACTG | |
| AAATTCTGAATGGATAGGACTCTGGAACATTCTCAATAGTGAGGGAGTTCTTTTTTTTTTTTTTTTTTTTTTAACTTA | |
| ACTGTCAACT | |
| Sequence 4: NbPDF1.2 terminator DNA sequence | |
| >NbPDF1.2 term [Niben101Scf00761g00006.1] | |
| TAATTGAAATTGGATTGATGCTATGTATGCCCAAAAAAGGGAGATATGTGAAGTTTGTTATAATAAGGCGAGAGATGT | |
| AAATTACACCAATAGGTGTAAGACTTCTTTATATTCTCGGTATGTATAACTTAAATCATCGTGATAGTGCACTCAAAC | |
| TAAGTCTCTTCCATAAGAATAACAAATAAATAAATAAATAAATAAAATCTTTTTTTCTTTTTGTCCAAGAAAATAAAT | |
| AAAAACTCCACATACGCGACACGATTGCCTTGACAAATGGAAGAATATTGAAGGTAGCATGCATGTGAGGCAATCTTG | |
| ACGACTTCAAAAACTTCTTAAGATGTTGAATGGCATCAGAAATAACATTTTAGTATGTGTTTACATAAGTTTCATCAC | |
| TTTGCCTAAAAACAAGGAAGTTGTGAGTTCGAGTCATTCTAAGAGCAAGGTGGAGAGTTCTTCAAAGGAAGAATGTCG | |
| GGGATCTATTTGGAAACAACCTATCTACCCTATGATACGGGTAAGGTTTGCGTATACACTATTCTTCCAGATCCCACT | |
| TTTAAAATTATACTAGCTTGTTATTATATTTAATTATGTTATAAATTTAAGTAATTAAAGAAAGACTTTTGACATCTA | |
| AGTCTAGCA | |
| Sequence 5: StPINII terminator DNA sequence | |
| >potato PIN-II [ST4.03ch03:50056201-50058200] | |
| CCCTAGACTTGTCCATCTTCTGGATTGGCCAACTTAATTAATGTATGAAATAAAAGGATGCACACATAGTGACATGCT | |
| AATCACTATAATGTGGGCATCAAAGTTGTGTGTTATGTGTAATTACTAATTATCTGAATAAGAGAAAGAGATCATCCA | |
| TATTTCTTATCCTAAATGAATGTCACGTGTCTTTATAATTCTTTGATGAACCAGATGCATTTTATTAACCAATTCCAT | |
| ATACATATAAATATTAATCATATATAATTAATATCAATTGGGTTAGCAAAACAAATCTAGTCTAGGTGTGTTTTGCTA | |
| ATTATTGGGGGATAGTGCAAAAAGAAATCTACGTTCTCAATAATTCAGATAGAAAACTTAATAAAGTGAGATAATTTA | |
| CATAGATTGCTTTTATCCTTTGATATATGTGAAACCATGCATGATATAAGGAAAATAGATAGAGAAATAATTTTTTAC | |
| ATCGTTGAATATGTAAACAATTTAATTCAAGAAGCTAGGAATATAAATATTGAGGAGTTTATGATTA | |
| Sequence 6: Tnos terminator DNA sequence | |
| >CP032929.1 Agrobacterium tumefaciens | |
| strain 1D1460 plasmid pTi1D1460, complete | |
| sequence | |
| AGAAGGAGTGCGTCGAAGCAGATCGTTCAAACATTTGGCAATAAAGTTTCTTAAGATTGAATCCTGTTGCCGGTCTTG | |
| CGATGATTATCATATAATTTCTGTTGAATTACGTTAAGCATGTAATAATTAACATGTAATGCATGACGTTATTTATGA | |
| GATGGGTTTTTATGATTAGAGTCCCGCAATTATACATTTAATACGCGATAGAAAACAAAATATAGCGCGCAAACTAGG | |
| ATAAATTATCGCGCGCGGTGTCATCTATGTTACTAGATCGATCAAACTTCGGCACTGTGTAATGACGATGAGCAATCG | |
| AGAGGCTGACTAACAAAAGGTATGCCCAAAAACAACCTCTCCAAACTGTTTCGAATTGGAAGTTTCTGCTCATGCCGA | |
| CAGGCATAACTTAGATATTCGCGGGCTATTCCCACTAATTCGTCCTGCTGGTTTGCGCCAAGATAAATCAGTGCATCT | |
| CCTTACAAGTTCCTCTGTCTTGTGAAATGAACTGCTGACTGCCCCCCAAGAAAGCCTCCTCATCTCCCAGTTGGCGGC | |
| GGCTG | |
| Sequence 7: AtMAR1a DNA sequence | |
| AGAGTTGCCGGTGCAACTCCTCCCATACCAAAATAGTGTTTTATCTTATTTTTAAAATAGATAACTTTTTCAACTTAT | |
| TTGTTTAAAATTTTAGCTTACGTTACATGTTATAATAATAATTAAAAAAAGAAGAAGATATAGTAATAACATTTGAGA | |
| AGTTGCATTTGGGTTCCATGTTTATACATTCTTCACTTCTAATGAATAACACCTTGAAACATCGAGTCCATAATTGAA | |
| TTTAGTCTTACAATGTTACAGACCTTCAAATTGTTTCCATAAATCTTTGAAATATGTCATAACACAAAATCTGAGAGT | |
| GGTTTATAATTTTATTAGATAAGACTCGTCCAAATATGCTGGTGTGAGAAATGTTGTGATGATATAAAAATTCTAAAT | |
| AGTAATCGGTATTTTTCAAAAGAATATTTCAATTATATACATTCCATATACCATGCCTTTACTAATTATATATAATAC | |
| GGGATTTCTCAAAGTATAAAAGAAAGATAAGACAAGGAAAACATACAACATCTATAAAAGACGCACCAGACAGAGCCT | |
| CTCATTGAATTTTGTGGTAAACGAATAGAACTCTGTAATTGAGAAAAAAATAAAATGAAATAATATTAAGAAATAAAA | |
| AGAGAGACATATTTAAGTTACAAAATTAGAAAATATTAAATCACGTATTAATATTTTTCTAAAACAAAATAGGGAACT | |
| TTTACTTCTGTTTTGAATATATAAACGATTTTTAATAGAATTTGTTCAATATTTTAATATAGTAAATAAAAAATTTGT | |
| ATATATGAACTATTTTTTTTACTTGTGTATATATCCGATGGGGTTATGAAATTTTTCTATATTTTGTTTTTTAAAAAT | |
| ATAGGGTTTTGTGCAAAACACCCATCTAACTCATCTAGTTTTGCAATTTTACCCTATATCTCTAACAAACACATATTA | |
| ATCTTATGAAGTCTAAAATAATACGCAATATGACCCTTATACGTATATTGTAAACTTTTTTACACTCCCATAGATTAG | |
| ATTTTTAATATTTCGAAGGTCGATTTGAACTTTTCGTCGAGAAAGCTTCGATTAAACATATTAAATATCAATATCTTA | |
| CAAAATATTGTCGTAGAAAAATGACGAAAAGGGGTTAAATTGCGTATTATTTTAGACTTCATCGGATAATTATGCATT | |
| TTTTGGAGGTGGAGAATAAAGATGCAAAAATAGATGAGTTAGAGGGTTTTATTTTGCACAAAACCCTAAAAATAACAA | |
| AATTTGATTTGATAAGGGTGGATATTTCACTAATAAATTTGACACGTGTCATGATCACGTGAGTTAACAATTTTGAAA | |
| GCCCAACTTTATGTAGATATTTGGCACATATATTGGATTATGTTCATTGTTTTCATGCCAGATCCGAACTGCTCTTCC | |
| CAACCGTTTAACCCGCTATCCAGAAACTCTTCTCCTCTAAGATTAGTGTTAACAAGTCATGGAGAACCTCATTGTATA | |
| GAAGTTTTACAAACTAAACCTTTAGATTGATTCAAATAATTTCACGAAGAAAATATTATAAATCGATTTATTTGTTTA | |
| AATTTGGAGATATAAACCGATGATTGATTAATTATGTGAAGCCATTTTACTTTTATTTTCTCTTTGTAGTCAATATTT | |
| CTTAGTTAATGTTACGTGTTTCACACATGGAGTTTCAACTATATTAAATTTCTAAAACGGCTAATCAAATACCTAATT | |
| AACGTATTCAAAGATGTTAAATTAAAAAAAATTAATATGACAGTTTCACATTTCAATCCATATAAATAAGCAAGGATT | |
| TTATTGTTATTCATCAATCAAAAACAAACGAAGCATTAAGCATATAGTTGCTATACTAAGACTTTTTACTCAATTTCT | |
| CATTATGTCTATCAAAAATGTGTTTTCACTTCTAGCGGTTCTATGTATCA | |
| Sequence 8: NtPR1a protein sequence | |
| >NtPR1a CAA31233.1 unnamed protein product | |
| [Nicotiana tabacum] | |
| MGFVLFSQLPSFLLVSTLLLFLVISHSCRAQNSQQDYLDAHNTARADVGVEPLTWDDQVAAYAQNYASQLAADCNLVH | |
| SHGQYGENLAEGSGDFMTAAKAVEMWVDEKQYYDHDSNTCAQGQVCGHYTQVVWRNSVRVGCARVQCNNGGYVVSCNY | |
| DPPGNYRGESPY | |
| Sequence 9: NbPINII protein sequence | |
| >NbPINII Niben101Scf07757g00001.1 protein | |
| sequence | |
| MAVHKVSFLAHLLVLGIFLLLVDAKACTKECGNFAYGRCPRSQGTPDDPICTSCCAGYKGCNYYNANGTFICEGLSDP | |
| RKPNFCPLFCDPDIAYSKCPRSEGETIINPTGCTTCCTGYKGCYYFGQDGKFVCEGESDEPKSCTTECDPRVAYISCP | |
| ISGLVKINQECINCCNADKGCELYDNDGSLICTGGEPQSAA | |
| Sequence 10: NbWIN1 protein sequence | |
| >NbWIN1 (NbPR4) Niben101Scf01015g01002.1 | |
| protein sequence | |
| MGKLSTVLLALILFIIAAGANAQQCGRQRGGALCGGNLCCSQFGWCGSTPEYCSPSQGCQSQCSGGGGGGGGGGGSAQ | |
| NVRATYHIYNPQNVGWDLYAVSAYCSTWDGNKPLAWRRKYGWTAFCGPVGPRGRDSCGKCLRVTNTGTGAQTTVRIVD | |
| QCSNGGLDLDVNVFRQLDTDGRGNQRGHLIVNYEFVNCGDNMNVLVSPVDKE | |
| Sequence 11: NbPDF1.2 protein sequence | |
| >NbPDF1.2 Niben101Scf00761g00006.1 protein | |
| sequence | |
| MAKSQIQCAVFLALFFCFLVASTEMMQKAEALGCEKPSTTWSGPCLFSDDCNNQCINWEGAVHGECSFALGPECFCYF | |
| C | |
| Sequence 12: Expression vector with StPINII | |
| NtPR1a - AtMAR1a DNA sequence | |
| >2x35S-TEV omega - MCS - StPINII-NtPR1a - | |
| AtMAR1a | |
| CAACATGGTGGAGCACGACACTCTCGTCTACTCCAAGAATATCAAAGATACAGTCTCAGAAGACCAAAGGGCTATTGA | |
| GACTTTTCAACAAAGGGTAATATCGGGAAACCTCCTCGGATTCCATTGCCCAGCTATCTGTCACTTCATCAAAAGGAC | |
| AGTAGAAAAGGAAGGTGGCACCTACAAATGCCATCATTGCGATAAAGGAAAGGCTATCGTTCAAGATGCCTCTGCCGA | |
| CAGTGGTCCCAAAGATGGACCCCCACCCACGAGGAGCATCGTGGAAAAAGAAGACGTTCCAACCACGTCTTCAAAGCA | |
| AGTGGATTGATGTGATAACATGGTGGAGCACGACACTCTCGTCTACTCCAAGAATATCAAAGATACAGTCTCAGAAGA | |
| CCAAAGGGCTATTGAGACTTTTCAACAAAGGGTAATATCGGGAAACCTCCTCGGATTCCATTGCCCAGCTATCTGTCA | |
| CTTCATCAAAAGGACAGTAGAAAAGGAAGGTGGCACCTACAAATGCCATCATTGCGATAAAGGAAAGGCTATCGTTCA | |
| AGATGCCTCTGCCGACAGTGGTCCCAAAGATGGACCCCCACCCACGAGGAGCATCGTGGAAAAAGAAGACGTTCCAAC | |
| CACGTCTTCAAAGCAAGTGGATTGATGTGATATCTCCACTGACGTAAGGGATGACGCACAATCCCACTATCCTTCGCA | |
| AGACCCTTCCTCTATATAAGGAAGTTCATTTCATTTGGAGAGGACACGCTGAAATCACCAGTGTATTTTTACAACAAT | |
| TACCAACAACAACAAACAACAAACAACATTACAATTACTATTTACAATGGCGCGCCCCCTAGACTTGTCCATCTTCTG | |
| GATTGGCCAACTTAATTAATGTATGAAATAAAAGGATGCACACATAGTGACATGCTAATCACTATAATGTGGGCATCA | |
| AAGTTGTGTGTTATGTGTAATTACTAATTATCTGAATAAGAGAAAGAGATCATCCATATTTCTTATCCTAAATGAATG | |
| TCACGTGTCTTTATAATTCTTTGATGAACCAGATGCATTTTATTAACCAATTCCATATACATATAAATATTAATCATA | |
| TATAATTAATATCAATTGGGTTAGCAAAACAAATCTAGTCTAGGTGTGTTTTGCTAATTATTGGGGGATAGTGCAAAA | |
| AGAAATCTACGTTCTCAATAATTCAGATAGAAAACTTAATAAAGTGAGATAATTTACATAGATTGCTTTTATCCTTTG | |
| ATATATGTGAAACCATGCATGATATAAGGAAAATAGATAGAGAAATAATTTTTTACATCGTTGAATATGTAAACAATT | |
| TAATTCAAGAAGCTAGGAATATAAATATTGAGGAGTTTATGATTATTGAAACGACCTACGTCCATTTCACGTTAATAT | |
| GTATGGATTGTTCTGCTTGATATCAAGAACTTAAATAATTGCTCTAAAAAGCAACTTAAAGTCAAGTATATAGTAATA | |
| GTACTATATTTGTAATCCTCTGAAGTGGATCTATAAAAAGACCAAGTGGTCATAATTAAGGGGAAAAATATGAGTTGA | |
| TGATCAGCTTGATGTATGATCTGATATTATTATGAACACTTTTGTACTCATACGAATCATGTGTTGATGGTCTAGCTA | |
| CTTGCGATATTACGAGCAAAATTCTTAACTACATGCCTTAGGAACAAGCTTACACAGTTCATATAATCTACTAGAGGG | |
| CCAAAAACATGAAAATTACCAATTTAGATGGTAGGAGGATATTGAAAGTGGAGCAGCTAGTTTTAATAACTGACCGTT | |
| AGTCTTAAAATTGACGGTATAAAAATATTTACATAATCAGGTCATTTATAAGGTAATTATAGGTAAATATTTATGACG | |
| AATTCTCAATAGTAATCTGAAAAAAAATTGTAACTAACCTATTATACTAAAACTACTATAATAGGTTAGATTACATTA | |
| ATCATGTCATTAGAAGATCTTAGCTCGGTACCCGGGGATCCTCTAGAGTCGACCTGCAGGCATGCAGAGTTGCCGGTG | |
| CAACTCCTCCCATACCAAAATAGTGTTTTATCTTATTTTTAAAATAGATAACTTTTTCAACTTATTTGTTTAAAATTT | |
| TAGCTTACGTTACATGTTATAATAATAATTAAAAAAAGAAGAAGATATAGTAATAACATTTGAGAAGTTGCATTTGGG | |
| TTCCATGTTTATACATTCTTCACTTCTAATGAATAACACCTTGAAACATCGAGTCCATAATTGAATTTAGTCTTACAA | |
| TGTTACAGACCTTCAAATTGTTTCCATAAATCTTTGAAATATGTCATAACACAAAATCTGAGAGTGGTTTATAATTTT | |
| ATTAGATAAGACTCGTCCAAATATGCTGGTGTGAGAAATGTTGTGATGATATAAAAATTCTAAATAGTAATCGGTATT | |
| TTTCAAAAGAATATTTCAATTATATACATTCCATATACCATGCCTTTACTAATTATATATAATACGGGATTTCTCAAA | |
| GTATAAAAGAAAGATAAGACAAGGAAAACATACAACATCTATAAAAGACGCACCAGACAGAGCCTCTCATTGAATTTT | |
| GTGGTAAACGAATAGAACTCTGTAATTGAGAAAAAAATAAAATGAAATAATATTAAGAAATAAAAAGAGAGACATATT | |
| TAAGTTACAAAATTAGAAAATATTAAATCACGTATTAATATTTTTCTAAAACAAAATAGGGAACTTTTACTTCTGTTT | |
| TGAATATATAAACGATTTTTAATAGAATTTGTTCAATATTTTAATATAGTAAATAAAAAATTTGTATATATGAACTAT | |
| TTTTTTTACTTGTGTATATATCCGATGGGGTTATGAAATTTTTCTATATTTTGTTTTTTAAAAATATAGGGTTTTGTG | |
| CAAAACACCCATCTAACTCATCTAGTTTTGCAATTTTACCCTATATCTCTAACAAACACATATTAATCTTATGAAGTC | |
| TAAAATAATACGCAATATGACCCTTATACGTATATTGTAAACTTTTTTACACTCCCATAGATTAGATTTTTAATATTT | |
| CGAAGGTCGATTTGAACTTTTCGTCGAGAAAGCTTCGATTAAACATATTAAATATCAATATCTTACAAAATATTGTCG | |
| TAGAAAAATGACGAAAAGGGGTTAAATTGCGTATTATTTTAGACTTCATCGGATAATTATGCATTTTTTGGAGGTGGA | |
| GAATAAAGATGCAAAAATAGATGAGTTAGAGGGTTTTATTTTGCACAAAACCCTAAAAATAACAAAATTTGATTTGAT | |
| AAGGGTGGATATTTCACTAATAAATTTGACACGTGTCATGATCACGTGAGTTAACAATTTTGAAAGCCCAACTTTATG | |
| TAGATATTTGGCACATATATTGGATTATGTTCATTGTTTTCATGCCAGATCCGAACTGCTCTTCCCAACCGTTTAACC | |
| CGCTATCCAGAAACTCTTCTCCTCTAAGATTAGTGTTAACAAGTCATGGAGAACCTCATTGTATAGAAGTTTTACAAA | |
| CTAAACCTTTAGATTGATTCAAATAATTTCACGAAGAAAATATTATAAATCGATTTATTTGTTTAAATTTGGAGATAT | |
| AAACCGATGATTGATTAATTATGTGAAGCCATTTTACTTTTATTTTCTCTTTGTAGTCAATATTTCTTAGTTAATGTT | |
| ACGTGTTTCACACATGGAGTTTCAACTATATTAAATTTCTAAAACGGCTAATCAAATACCTAATTAACGTATTCAAAG | |
| ATGTTAAATTAAAAAAAATTAATATGACAGTTTCACATTTCAATCCATATAAATAAGCAAGGATTTTATTGTTATTCA | |
| TCAATCAAAAACAAACGAAGCATTAAGCATATAGTTGCTATACTAAGACTTTTTACTCAATTTCTCATTATGTCTATC | |
| AAAAATGTGTTTTCACTTCTAGCGGTTCTATGTATCA | |
| 2x35S promoter | 1 . . . 750 |
| TEV omega | 751 . . . 828 |
| MCS (to insert gene of interest) | 829 . . . 836 |
| StPINII | 837 . . . 1371 |
| NtPR1a | 1372 . . . 1971 |
| AtMAR1a | 2016 . . . 3937 |

1-23. (canceled)

24. An expression vector for use in transforming a plant or plant cell comprising a nucleic acid sequence of sequence ID no 7 comprising a matrix attachment region or a fragment of at least 300 base pairs or variant having 70% identity to said region or fragment.

25. An expression vector as claimed in claim 24, additionally comprising a terminator sequence selected from SEQ ID NO:1, SEQ ID NO: 2, SEQ ID NO: 3 and SEQ ID NO: 4, a terminator homologue thereof, or a fragment of said sequence, homologue, or variant having 80% sequence identity to SEQ ID NO:1, SEQ ID NO: 2, SEQ ID NO: 3 or SEQ ID NO: 4 homologue or fragment.

26. An expression vector as claimed in claim 24, additionally comprising a second terminator sequence.

27. An expression vector as claimed in claim 26, comprising two terminator sequences selected from sequence ID No 1 to 6, homologues, variants, and fragments of said sequence ID No 1-6, and terminators having 80% Identity to said sequence ID No 1-6, homologues, variants and fragments.

28. An expression vector as claimed in claim 24, comprising two matrix attachment region sequences said sequences selected from: sequence ID No 7, a fragment of at least 300 base pair of sequence ID no 7, or variant having 70% identity to said region or fragment.

29. An expression vector as claimed in claim 24, comprising an open reading frame sequence encoding a polypeptide.

30. An expression vector as claimed in claim 34 comprising a promoter.

31. An expression vector as claimed in claim 24, additionally comprising a silencing suppressor.

32. An expression vector as claimed in claim 36, wherein the silencing suppressor is P19.

33. An expression vector as claimed in claim 24, the expression vector comprising an expression cassette comprising either a single or tandem terminator and wherein the matrix attachment region is either down stream of the expression cassette, upstream of the expression cassette or is both upstream and downstream of the expression cassette.

34. An expression vector as claimed in claim 24, the expression vector comprising an expression cassette comprising either a single or tandem terminator and wherein the matrix attachment region is located either at the 5′UTR of the expression cassette, at the 3′UTR of the expression cassette or at both the 5′ and 3′ UTRs of the expression cassette.

35. An expression vector as claimed in claim 24 which comprises a tandem terminator and wherein the matrix attachment region is downstream of the expression cassette.

36. An expression vector comprising the following components in the following 5′ to 3′ order:
a promoter and optionally, a second promoter
a polypeptide encoding open reading frame
a terminator of sequence ID No 5
a terminator of sequence of sequence ID no 6
a matrix attachment region of sequence ID No 7.

37. An expression vector comprising the following components in the following 5′ to 3′ order:
a double CaMV 35S promoter
a tobacco etch virus omega 5′UTR (TEV omega)
a polypeptide encoding open reading frame
a terminator of sequence ID No 5
a terminator of sequence of sequence ID no 6
a matrix attachment region of sequence ID No 7.

38. An expression vector comprising the following components in the following 5′ to 3′ order
a promoter and optionally a second promoter
a polypeptide encoding open reading frame
a terminator of sequence ID No 6
a terminator of sequence of sequence ID no 1
a matrix attachment region of sequence ID No 7.

39. An expression vector comprising the following components in the following 5′ to 3′ order:
a promoter and optionally, a second promoter
a polypeptide encoding open reading frame
a terminator of sequence ID No 5
a terminator of sequence of sequence ID no 6
a matrix attachment region of sequence ID No 7.

40. A host transformed with the vector as claimed claim 24.

41. A host as claimed in claim 40, wherein the host is derived from Viridiplantae.

42. A method of expression of a protein in a plant or plant cell comprising transforming a plant host cell with a vector as claimed in claim 24, expressing said protein and collecting said protein.

43. A method of claim 42, wherein said expression is transient.
